(19) United States
(12) Patent Application Publication
(10) Pub. No.: US 2023/0120782 A1
(43) Pub. Date: Apr. 20, 2023

(54) PHOTOTHERAPY AND PHOTOBIOMODULATION DEVICE

(71) Applicant: Lumitex, Inc., Strongsville, OH (US)

(72) Inventors: Anton Zonneveld, Strongsville, OH (US); Alan Greszler, Strongsville, OH (US); Michael Kerns, Strongsville, OH (US); Carolyn Guzik, Strongsville, OH (US); Dinusha Thotagamuwa, Strongsville, OH (US)

(21) Appl. No.: 17/906,203

(22) PCT Filed: Jun. 11, 2021
(86) PCT No.: PCT/US2021/036901

§ 371 Date: Sep. 13, 2022

Related U.S. Application Data
(60) Provisional application No. 63/042,590, filed on Jun. 23, 2020.

(51) Int. Cl. A61N 5/06

(52) U.S. Cl. CPC A61N 5/0622; A61N 2005/0663; A61N 2005/0659; A61N 2005/0648

(57) ABSTRACT

A photobiomodulation (PBM) device including eyewear (e.g., incorporating prescription or non-prescription lenses) that is worn throughout the day is provided. The PBM device provides longer-term light delivery throughout the day and may capture patient compliance, provide real-time dose adjustments, and allow for HIPAA-compliant communication with the prescribing clinician. The PBM device provides a wearable, open eye device format designed for increased compliance, lower cost, and greater convenience.

### TECHNICAL FIELD

The present disclosure relates generally to phototherapy and more particularly to using phototherapy for treatment of eye trauma.

### BACKGROUND

Diabetic macular edema (DME) is the most common cause of vision loss among the Diabetic Retinopathy (DR) patient population. DME is a condition that develops due to disrupted inner or outer blood retinal barriers (BRB). Diabetic retina produces free radicals (oxidative stress), inflammatory mediators and increased levels of vascular endothelial growth factor (VEGF), cytokines and chemokines leading to disruption of BRB. This creates an imbalance between the fluid entry and exit resulting in an excessive fluid volume in the macula. Consequently unbalanced hydration state in the macula affects the tissue transparency and light transmission impairing the vision.

In the US alone 34.1 million adults have Diabetes Mellitus. According to the Diabetes Control and Complications Trial (DCCT), the incidence of DME in type I and type II diabetic patients is 27% within 9 years of onset. Nearly 7.7 million people in the US over the 40 years of age suffer from DR and that 750,000 individuals in the same population have DME and it is growing by 75,000 each year. Center-Involving DME (CI DME) develops to moderate visual loss within 3 years with no treatment. In the US, individuals with DME incur 30% higher medical costs ($8,000) than healthy adults in the first 3 years of incidence imposing a substantial burden on the healthcare systems. Furthermore, DME-induced vision impairment degrades the quality of life and deprives their living independently.

Current treatment approaches to DME are twofold: (1) lifestyle changes to address the underlying diabetic condition; and (2) possible combinations of laser photocoagulation, intravitreal corticosteroids, and intravitreal anti-Vascular Endothelial Growth Factor (VEGF) injections. Approximately 13% of the patients with CI DME in tight blood sugar and blood pressure control regime reduced their visual acuity (VA) after 2-3 years follow-up. Besides the many undesirable side effects of laser photocoagulation, the treatment often has a poor efficacy against diffuse DME. Though intravitreal anti-VEGF injections are currently the first line of treatment, this treatment plan requires recurrent injections as frequently as monthly and lasting for many years which is inconvenient for patients and their families. In summary, current interventions for DME are less efficacious, invasive, inconvenient, expensive, and associated with unwanted side effects.

Photobiomodulation (PBM) is a form of light therapy that utilizes non-ionizing form of light sources, e.g., in the visible and infrared (IR) spectrum. PBM in the far red and IR range (660-850 nm) has been identified as a safe, non-invasive, and non-pharmacological treatment for DME through demonstrated anatomical and functional improvement in the macula.

PBM is based on modulation of the activity of cytochrome C oxidase, a photoreceptor in the mitochondria. Previous studies have shown that PBM can increase the mitochondrial metabolism and production of cytoprotective factors, decrease inflammation and prevent cell death. PBM treatment has demonstrated the successful resolution of several retinal diseases including DR, Age-related Macular Degeneration (AMD) and Retinopathy of Prematurity (ROP) in animal models. PBM involves several metabolic pathways that terminate in countering inflammatory cell migration, anti-apoptotic, and anti-oxidative events in a damaged retina to resolve the disease.

### SUMMARY

The existing photobiomodulation (PBM) devices for diabetic macular edema (DME) use LED arrays or lasers. These PBM devices are mainly used in a high resource setting and require clinician oversight to administer the light therapy. These devices are not portable and patients need to be immobilized during the treatment. Daily visits, clinician oversight, and inconvenience render this intervention less compliant and acceptable. A key disadvantage of this method is light therapy is administered to the closed eye which makes the accurate dose control more difficult with the variability of eyelid transmittance among individuals.

In a general embodiment, the present disclosure provides a photobiomodulation (PBM) device including eyewear (e.g., incorporating prescription or non-prescription lenses) that is worn throughout the day. The PBM device provides longer-term light delivery throughout the day and may capture patient compliance, provide real-time dose adjustments, and allow for HIPAA-compliant communication with the prescribing clinician. The PBM device provides a wearable, open eye device format designed for increased compliance, lower cost, and greater convenience.

While a number of features are described herein with respect to embodiments of the invention; features described with respect to a given embodiment also may be employed in connection with other embodiments. The following description and the annexed drawings set forth certain illustrative embodiments of the invention. These embodiments are indicative, however, of but a few of the various ways in which the principles of the invention may be employed. Other objects, advantages and novel features according to aspects of the invention will become apparent from the following detailed description when considered in conjunction with the drawings.

### BRIEF DESCRIPTION OF THE DRAWINGS

The annexed drawings, which are not necessarily to scale, show various aspects of the invention in which similar reference numerals are used to indicate the same or similar parts in the various views.

FIG. 1 is an image of an exemplary embodiment of the phototherapy device having a power source located within frames.

FIG. 2 is an image of another exemplary embodiment of the phototherapy device having a power source located outside of the frames.

FIG. 3 is an image of a further exemplary embodiment of the phototherapy device having a power source located outside of the frames.

FIG. 4 is an image of the phototherapy device of FIG. 3 showing light emission from optical lenses.

FIG. 5 is a schematic diagram of an eye showing an area illuminated by electromagnetic radiation emitted by the phototherapy device.

FIG. 6 shows an exemplary embodiment of an optical lens having light-extracting features.

FIG. 7 is a zoomed in view of the light-extracting features of the optical lens of FIG. 5.

FIG. 8 is a perspective view of a charger for the phototherapy device of FIG. 2.

FIG. 9 is another perspective view of the charger of FIG. 8.

FIG. 10 is a top view of the charger of FIGS. 8 and 9.

FIG. 11 is a perspective view of a charger for the phototherapy device of FIG. 1.

FIG. 12 is another perspective view of the charger of FIG. 11.

FIG. 13 is a top view of the charger of FIGS. 11 and 12.

The present invention is described below in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

### DETAILED DESCRIPTION

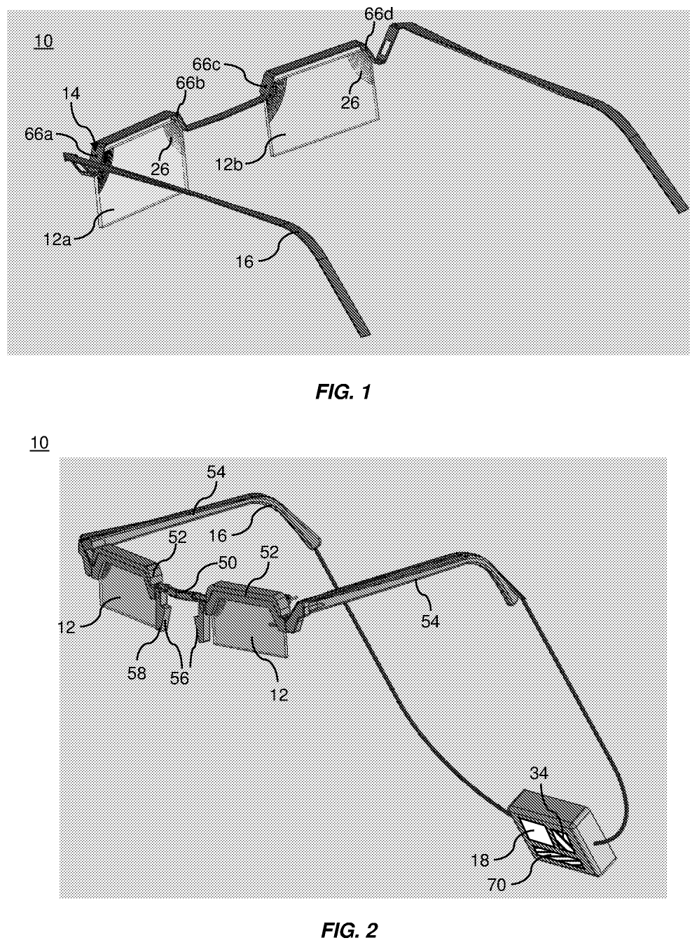

In the embodiment shown in FIG. 1, a photobiomodulation (PBM) device 10 (also referred to as a phototherapy device) is shown for delivering phototherapy to an eye of a user. The PBM device 10 includes an optical lens 12, a light source 14, frames 16, and circuitry 18. The light source 14 emits electromagnetic radiation 20. The frames 16 supports the optical lens 12 and the light source 14 relative to the eye. The optical lens 12 receives the emitted electromagnetic radiation 20 (e.g., along an edge 22 of the optical lens 12) and propagates the electromagnetic radiation 20 within the optical lens 12 via total internal reflection. The optical lens 12 includes light-extracting features 26 to extract the propagated electromagnetic radiation 20 from the optical lens 12, such that the extracted electromagnetic radiation 20 is directed in a pre-determined light distribution. The circuitry 18 determines an applied optical dose delivered by the extracted electromagnetic radiation 20. The circuitry 18 also controls the emission of the electromagnetic radiation 20 by the light source 14 based on both the determined applied optical dose and a defined optical dose.

In one embodiment, the applied optical dose is determined based on at least one of the time duration, intensity, and wavelength of the electromagnetic radiation. For example, the circuitry 18 may determine the applied dose based on the power supplied to the light source 14 and the period of time the power was supplied for. In one embodiment, the applied dose may be based on the watt hours or amp hours supplied to the light source 14. In this example, the defined optical dose may be specified in the same units (i.e., watt hour or amp hours), such that the determined applied optical dose may be directly compared to the defined optical dose.

The defined optical dose may be user specified, pre-defined (e.g., stored in the circuitry 18), etc. For example, the defined optical dose may depend on the trauma being treated. In one embodiment, the defined optical dose includes an illuminance of 0.4-0.8 mW/cm² at a cornea of the eye of the user. Below is a table showing exemplary optical doses for various diseases (treatments).

| | | | Irradiance/ | | Duration | |
|---|---|---|---|---|---|---|
| | | | Illuminance | | per | Total dose |
| Therapy Type | Wavelengths | | at cornea | | treatment | per day |
| Diabetic Macular | 670 | nm | 0.6 | mW/cm² | 2.5 h | 4-5 J/cm² |
| Edema (DME) | | | | | | |
| Age related | 660-670 | nm | 0.6 | mW/cm² | 2.5 h | 4-5 J/cm² |
| macular | | | | | | |
| degeneration | | | | | | |
| (AMD) | | | | | | |
| Diabetic | 670 | nm | 0.6 | mW/cm² | 2.5 h | 4-5 J/cm² |
| Retinopathy | | | | | | |
| (DR) | | | | | | |
| Circadian rhythm/ | 470 | nm | 10 | lux | 4 h | |
| Alzheimer | | | | | | |
| therapy | | | | | | |

Assuming a pupil diameter of 7 mm and total extraction efficiency of 25% from the optical system, the total light source output requirement may be calculated as follows for 670 nm:

$$\frac{0.6 mW / {cm}^{2} \times π \times {0.7}^{2} {cm}^{2}}{4 \times 025} = 0.92 mW .$$

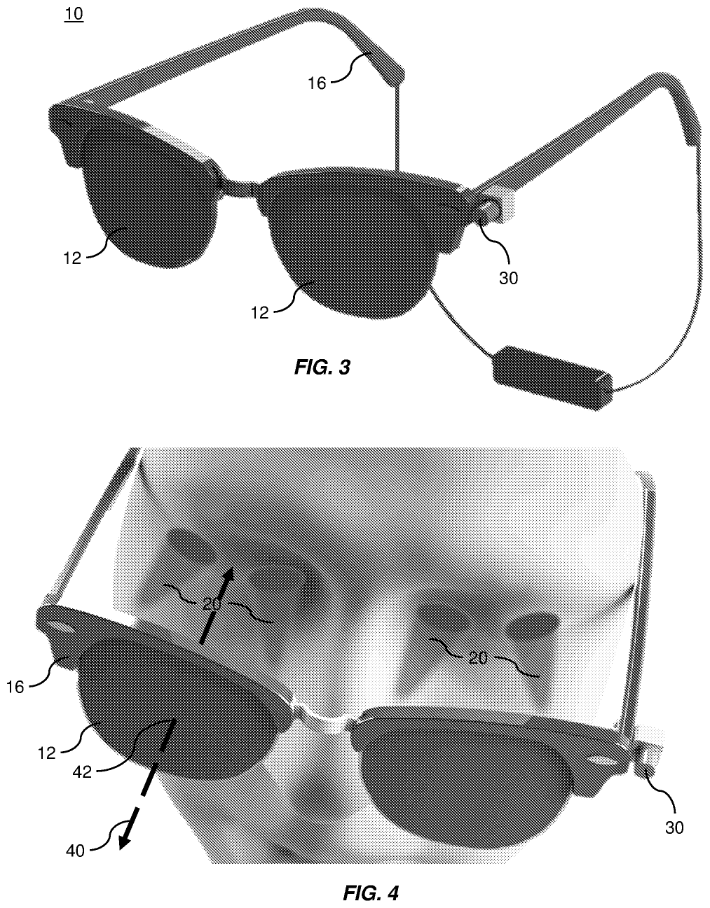

In the embodiment shown in FIG. 3, the PBM device 10 includes a photosensor 30. The photosensor 30 detects a property of ambient light. The circuitry 18 receives an output signal from the photosensor 30 based on the detected property. The ambient light may be the light from the external environment that is received by the eye of the user. For example, if a user is viewing a computer monitor, light from the computer monitor may be detected by the photosensor 30 as part of the ambient light. The circuitry 18 may control the emission of the electromagnetic radiation 20 based on the detected properties of the ambient light (in addition to the determined applied optical dose and the defined optical dose as described above).

In one embodiment, the detected property of the ambient light includes an intensity of the ambient light. When the detected intensity of the ambient light is below an ambient light intensity threshold, the emission of the electromagnetic radiation 20 may be controlled such that an intensity of the emitted electromagnetic radiation 20 is reduced below a therapeutic light intensity. For example, when a user is in a dark room, the intensity of the light emitted by the light source 14 may be reduced or the light source 14 may stop emitting light so that the user's perception in the dark room is not impeded by the electromagnetic radiation 20 from the light source 14.

In one embodiment, the photosensor 30 both receives a portion of the emitted electromagnetic radiation 20 and detects a property of the received portion of the electromagnetic radiation 20. The photosensor 30 may output a signal to the circuitry 18 based on the detected property of the received portion of the electromagnetic radiation 20. The circuitry 18 may determine the applied optical dose based on the detected property. The detected property may include at least one of wavelength or intensity. For example, the photosensor may detect an intensity of the electromagnetic radiation 20 and the circuitry 18 may use a sum of the detected intensity (e.g., across a time duration) to determine the applied optical dose. As an example, the photosensor 30 may periodically measure an intensity of the electromagnetic radiation 20. The circuitry 18 may determine the applied dose based on an integration of the measured intensity of the electromagnetic radiation 20.

In the embodiment shown in FIG. 2, the PBM device 10 also includes a monitoring sensor 34 that outputs a sensor measurement. For example, the monitoring sensor may include at least one of a current sensor or a temperature sensor. The current sensor outputs a current measurement as the sensor measurement based on a current supplied to the light source 14. The temperature outputs a temperature measurement as the sensor measurement based on at least one of a temperature of the light source 14 or a temperature of the eye of the user.

The circuitry 18 may determine the applied optical dose based on the sensor measurement. For example, the circuitry 18 may use a lookup table or a known relationship between the sensor measurement and an intensity or power of the electromagnetic radiation 20 output by the light source 14. Alternatively or additionally, the circuitry 18 may determine whether the sensor measurement is within an acceptable range and issue a notification when the sensor measurement is not within the acceptable range. For example, the circuitry 18 may emit as the notification an audible sound, a visual indicator (e.g., a light), and/or send a wireless notification (e.g., via a network).

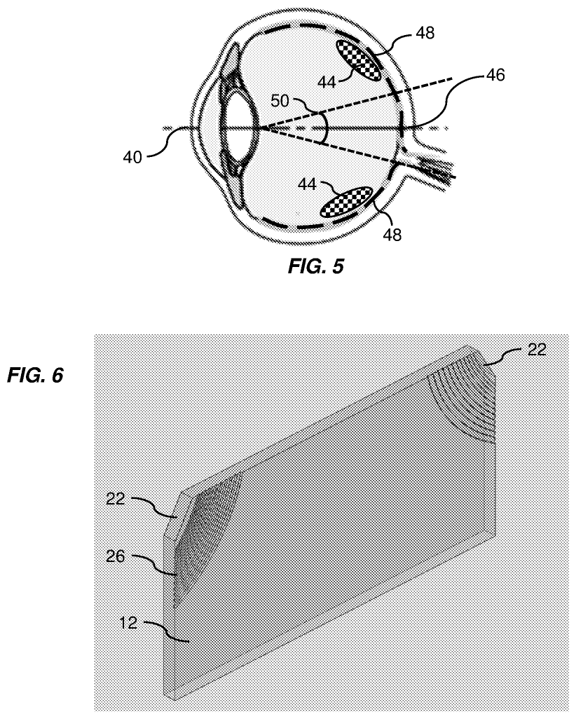

The pre-determined light distribution may describe the trajectory and relative intensity of the electromagnetic radiation 20 exiting the optical lens 12. In one embodiment, the optical lens 12 is configured to delivery therapeutic light to the back of the eye from the peripheral vision. That is, the pre-determined light distribution may have a trajectory that avoids the centra vision. In this way, the therapeutic light does not interfere with the user's direct line of sight and prevents obstruction of forward vision. For example, in FIGS. 4 and 5, the optical lens 12 has an optical axis 40 passing through a center of curvature 42 of the optical lens 12. The frames may be configured to position the optical lens 12 relative to the eye such that the optical axis 40 intersects with a retina of the eye. The light-extracting features 26 may be positioned away from the optical axis 40, such that the electromagnetic radiation 20 extracted by the light-extracting features 26 illuminates a peripheral portion 44 of the eye away from a fovea centralis 46 of the retina 48. For example, the peripheral portion 44 of the retina that is illuminated by the pre-determined light distribution may be outside of fifteen degrees 50 of the optical axis from the optical axis.

As described above, the frames 16 may support the optical lens 12, light source 14, and circuitry 18. In the embodiment shown in FIGS. 1-4, the frames 16 include a bridge 50, two rims 52, and two temples 54. The bridge 50 may have nose pads 56 that interact with a nose of the user when the frames are positioned on a face of the user. The two rims 52 include a support rim 58. Each of the two rims 52 is attached to the bridge 50 and the support rim 58 supports the optical lens 12. Each temple 54 may extend from one of the two rims 52 and interacts with an ear of the user when the frames are positioned on the face of the user.

The frames 16 may be made of any suitable material for supporting the optical lens 12. For example, the frames may include at least one of plastic or metal. The frames 16 may also be 3D printed or fabricated in any suitable manner.

In one embodiment, the light source 14 is physically supported by the support rim 58 adjacent to the edge 22 of the optical lens, such that the electromagnetic radiation 20 emitted by the light source 14 is received by the edge 22 of the optical lens 12. In another embodiment, the electromagnetic radiation 20 emitted by the light source 14 may be received by a light guide 62 and be transported by the light guide 62 to the edge 22 of the optical lens. The light guide 62 may emit the transported electromagnetic radiation, such that the electromagnetic radiation is received by the edge 22 of the optical lens 12. For example, the light guide 62 may include fiber optics or any suitable structure for transporting light via total internal reflection.

In one embodiment, the electromagnetic radiation 20 is delivered from the light source 14 via fiber bundles inserted through holes created in a top corner of the optical lens 12. The electromagnetic radiation 20 is propagated within the optical lens 12 before interacting with the light-extracting features 26 and being extracted from the optical lens 12.

The light source 14 may emit any suitable wavelength intensity of electromagnetic radiation. For example, the light source 14 may emit electromagnetic radiation in a wavelength range of 500 nm to 1000 nm. For example, over 50% of the electromagnetic radiation emitted by the light source 14 may be in the wavelength range of 650-670 nm. For example, the light source 14 may be a narrow band light source (e.g., having a full width half max (FWHM) of 20 nm) with a peak wavelength at 670 nm.

In one embodiment, the light source 14 includes multiple light emitters 66. The light emitters 66 may be any suitable structure for emitting electromagnetic radiation. For example, the light emitters 66 may include one or more light emitting diodes (LEDs), organic LEDs (OLEDs), microLEDs, laser diodes, mini-LED, quantum dot (QD)-conversion, phosphor conversion, excimer lamps, multi-photon combination, or SLM wavefront manipulation.

The light emitters 66 may be mounted to the frames 16 and/or optical lens 12 via any suitable method. For example, in the embodiment shown in FIGS. 1 and 2, the light emitters 66 may be mounted to a flexible printed circuit (FPC) and the FPC may be edge mounted to the optical lens 12.

In one embodiment, the optical lens 12 may be any suitable structure capable of receiving electromagnetic radiation along an edge and propagating the light within the optical lens via total internal reflection. For example, the optical lens 12 may be eye glass lenses with or without a prescription. As an example, the optical lens 12 may be without a prescription, such that light exiting the optical lens 12 is not concentrated or dispersed. Alternatively, the optical lens 12 may be shaped for concentrating or dispersing light exiting the optical lens 12. For example, the optical lens 12 may be custom ground to the user's prescription.

The optical lens 12 may be made of any suitable material. For example, the optical lens 12 may be made from glass or plastic. The optical lens 12 may also be transparent (e.g., partially transparent) to visible light. In one embodiment, the optical lens 12 is a flat polycarbonate lens. The optical lens 12 may attenuate blue light, such that a color of visible light passing through the optical lens 12 is red shifted.

In the embodiment shown in FIG. 1, the optical lens 12 includes a right lens 12a and a left lens 12b. The light source 14 may include four right lens light emitters 66a, 66b, and four left lens light emitters 66c, 66d. The four right lens light emitters may include a left side pair of light emitters 66b and a right side pair of light emitters 66a. Similarly, the four left lens light emitters may include a left side pair of light emitters 66d and a right side pair of light emitters 66c.

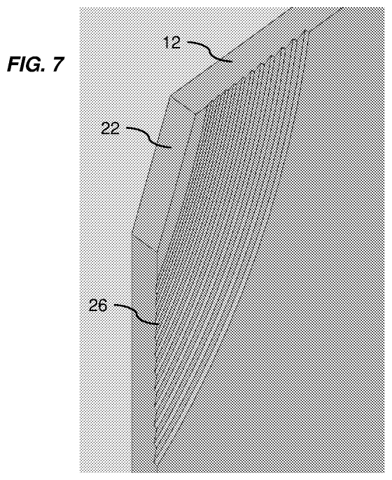

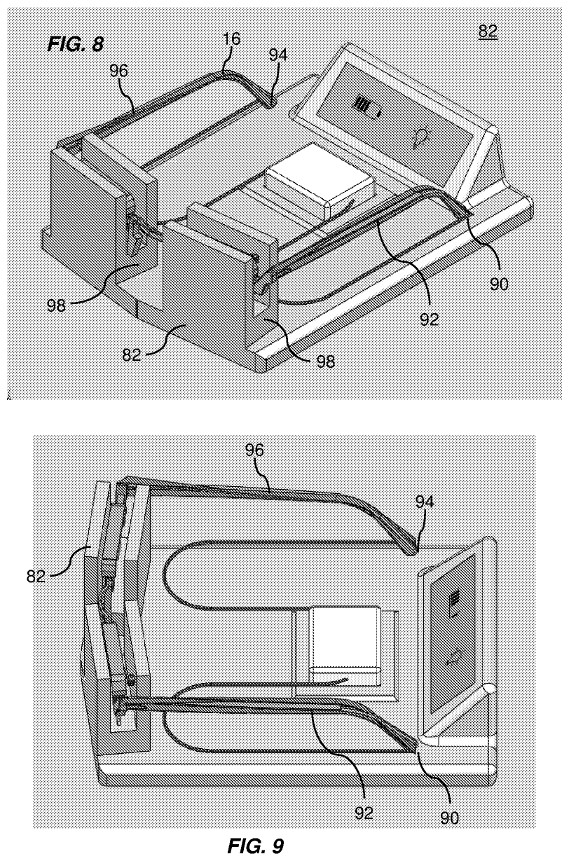

The light-extracting features 26 may include any suitable structures for extracting light from the optical lens 12 (e.g., to target the pre-determined light output distribution). For example, the light-extracting 26 features may include at least one of surface aberrations, micro-lenses, Fresnel pattern(s), stair step structures, reflective spots, partial reflective planes, or diffraction gratings. Alternatively or additionally, a diffuser sheet or a 2-D lensing sheet may be placed on an emission surface of the light guide. In one embodiment, the surface aberrations include at least one of a contour of the surface, surface depositions, or surface etchings. In the embodiment shown in FIGS. 6 and 7, the light-extracting features 26 include micro-lenses located adjacent the edge 22 at upper corners of the optical lens 12.

In one embodiment, the light-extracting features 26 include diffractive optics. Diffractive optics (e.g., for near-eye displays) use nanometer-scale elements that rely on wave diffraction to distribute light rather than total internal reflection (TIR) and refraction as microscale elements do. Using diffractive optics may allow for alternative locations of the light source, a lower profile of the optics, and decreased visibility of the optics.

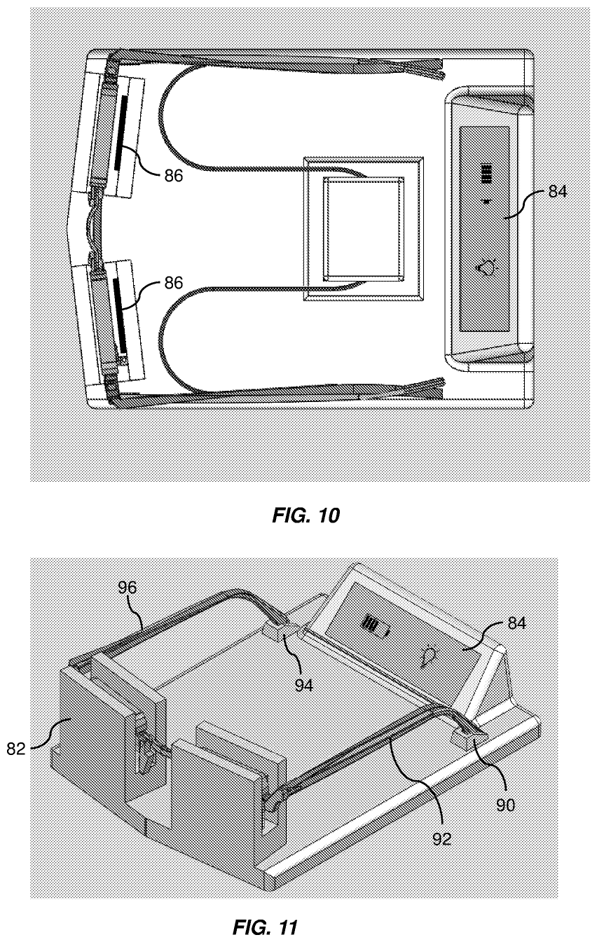

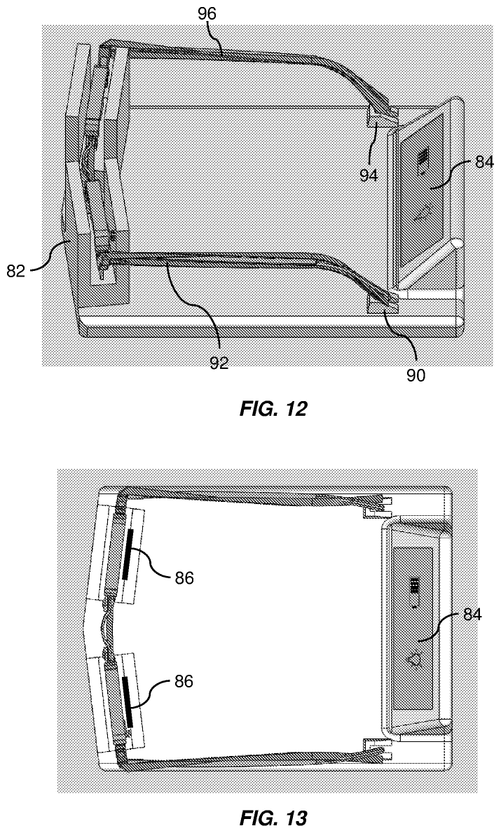

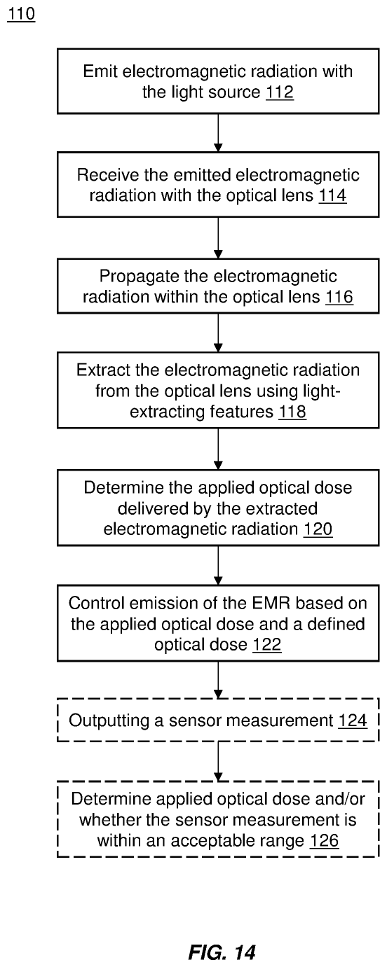

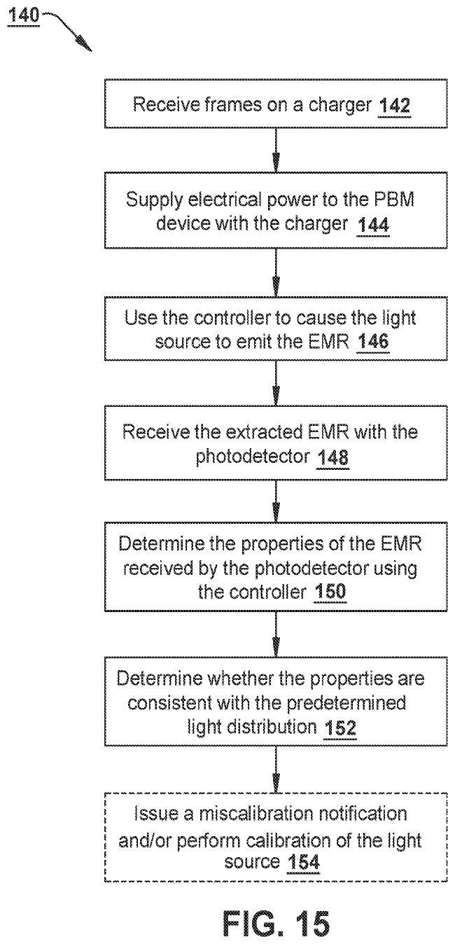

In the embodiments shown in FIGS. 8-13, the PBM device 10 includes a power storage device 70 physically supported by the frames 16. The power storage device 70 stores electrical power and supplies the stored electrical power to the circuitry 18 and the light source 14. For example, the power storage device 70 may be a rechargeable battery.

In the embodiment shown in FIGS. 11-13, the circuitry 18 and power supply 70 are housed in the frames 16 (e.g., to make the PBM device 10 lighter and more comfortable for long-time wear). The PBM 10 may also include a network interface (e.g., such as Bluetooth connectivity) for communicating with an electronic device. For example, the electronic device may be a mobile phone running an application that provides treatment parameters to the circuitry 18 (e.g., light irradiance, duration, etc.). The electronic device may also collect data and share information with clinicians (e.g., including data analytics and visualization), and send notification to remind the patient that treatment is required.

In the embodiment shown in FIG. 8, the PBM device 10 is included in a PBM system 80 having a charger 82. The charger 82 receives the frames 16 of the PBM device 10 and, when the frames 16 are received by the charger 82, supply the electrical power to the power storage device. The charger 82 includes a controller 84 and a photodetector 86. The controller 84 causes the light source 14 to emit the electromagnetic radiation 20. The photodetector 86 receives the electromagnetic radiation extracted from the optical lens 12. For example, the charger 82 may include an inductive charger for charging the power supply 70.

The controller 84 determines properties of the electromagnetic radiation received by the photodetector 86. For example, the controller 84 may determine a measured light distribution based on the electromagnetic radiation received by the photodetector 86. The controller 84 then determines whether the determined properties are consistent with the pre-determined light distribution. As an example, the controller 84 may determine a measured light distribution including an angular output of the electromagnetic radiation from the optical lens 12. The photosensor 86 may be positioned relative to the optical lens 12, such that when the frames 16 are positioned on the charger 82, the position of the photosensor 86 matches a location of a defined structure of the eye when the frames are positioned on the face of the user. For example, the photosensor 86 may be positioned where the user's cornea would be located, such that the photosensor 86 measures properties of the electromagnetic radiation that would be incident on the user's cornea if the user was wearing the PBM device 10.

When the measured light distribution is inconsistent with the pre-determined light distribution, the controller 84 may issue a miscalibration notification and/or perform calibration of the light source 14. For example, the controller 84 may perform calibration of the light source 14 until the measured light distribution is consistent with the pre-determined light distribution.

This calibration may include determining recalibration parameters based on the measured light distribution and the pre-determined light distribution. For example, if the intensity of the electromagnetic radiation is 10% lower than expected, the current supplied to the light source 14 by the controller 84 may be increased in an attempt to increase the intensity of the electromagnetic radiation to within a threshold of the expected value (e.g., within 1%, 3%, or 5% of the expected value). As an example, if the light intensity is 10% lower than expected, the current supplied to the light source may be increased by 10% or a lookup table or known relationship between supplied current and light intensity may be used to determine how much to increase the current by.

After determining the recalibration parameters, the recalibration parameters are issued to the circuitry 18. The circuitry 18 may then cause the light source 14 to emit electromagnetic radiation 20 based on the issued recalibration parameters, such that the photodetector 86 receives the electromagnetic radiation 20 extracted from the optical lens 12. The controller 84 may then determine the measured light distribution based on the electromagnetic radiation received by the photodetector 82. The controller 84 may then determine whether the measured light distribution is consistent with the pre-determined light distribution. If the measured light distribution is consistent with the pre-determined light distribution, then calibration may be stopped. Conversely, if the measured light distribution is noy consistent with the pre-determined light distribution, then the calibration may be performed again to determine a new set of recalibration parameters.

In one embodiment, the charger 80 also includes a left lateral support 90 for supporting a first temple 92 of the frames 16 and a right lateral support 94 for supporting a second temple 96 of the frames 16 when the frames 16 are supported by the charger 80. The charger 80 may also include a central support 98 for physically supporting the optical lens(es) 12. In one embodiment, instead of supporting the optical lens 12 directly, the central support 98 physically supports nose pads 56 of a bridge 50 of the frames 16 when the frames 16 are supported by the charger 80.

In the embodiment shown in FIG. 14, a method 110 is shown for treating eye trauma using a photobiomodulation (PBM) device to deliver phototherapy to an eye of a user. In step 112, electromagnetic radiation 20 is emitted with the light source 14. In step 114, the emitted electromagnetic radiation 20 is received along an edge of the optical lens 12. In step 116, the electromagnetic radiation 20 is propagated within the optical lens 12 via total internal reflection. In step 118, the propagated electromagnetic radiation 20 is extracted from the optical lens 12 using light-extracting features 26 of the optical lens 12, such that the extracted electromagnetic radiation is directed in a pre-determined light distribution. In step 120, an applied optical dose delivered by the extracted electromagnetic radiation is determined with the circuitry 18. In step 122, the emission of the electromagnetic radiation 20 by the light source 14 is controlled based on both the determined applied optical dose and a defined optical dose.

As described above, the applied optical dose may be determined based on at least one of the time duration, intensity, and wavelength of the electromagnetic radiation.

In one embodiment, the method 110 may additionally include (in step 124) outputting a sensor measurement with the monitoring sensor. As described above, the monitoring sensor may output a current measurement or a temperature measurement. In step 126, the circuitry determines the applied optical dose based on the sensor measurement or determines whether the sensor measurement is within an acceptable range. If the sensor measurement is not within the acceptable range, the circuitry may issue a notification.

In the embodiment shown in FIG. 15, a method 140 for charging and monitoring the output of the PBM device 10 is shown. In step 142, the frames 16 are received on a charger 82. In step 144, electrical power is supplied to the PBM device 10 with the charger 82. In step 146, the controller 84 of the charger 82 causes the light source 14 to emit the electromagnetic radiation 20. In step 148, the electromagnetic radiation extracted from the optical lens 12 is received with the photodetector 86. In step 150, properties of the electromagnetic radiation received by the photodetector is determined using the controller 84. In step 152, the controller 84 determines whether the determined properties are consistent with the pre-determined light distribution. As described above, the determined property may include a measured light distribution of electromagnetic radiation.

In optional step 154, when the measured light distribution is inconsistent with the pre-determined light distribution, a miscalibration notification may be issued with the controller or the controller may perform calibration of the light source until the measured light distribution is consistent with the pre-determined light distribution.

As described above, the trauma being treated using the PBM device 10 may be caused by at least one of diabetic retinopathy, macular degeneration, or diabetic macular edema. In one embodiment, the method may be used to treat trauma at a back of the eye.

In addition to sending notifications to caregivers, the circuitry 18 may also issue notifications to a user of the device 10. For example, when the power source level is below a threshold, the device 10 may vibrate to notify a user.

The device 10 may additionally include a GPS chip configured to determine a location of the device 10. For example, the GPS chip may be used to provide a location of a lost device 10 or of a user wearing the device 40.

The device 10 may include eye tracking. The eye tracking may be used to target particular areas of the eye. For example, a particular phototherapy may be targeted at a particular location on the eye. The device 10 may utilize eye tracking to ensure that only the particular location is illuminated with electromagnetic radiation 20 from the light source 14. In this way, the device 10 may conserve electrical power and reduce heat generation. The optical lens 12 may also be used to alter a beam width of light being directed towards the eye. The beam width may be controlled by the electronics depending on the type of therapy being applied, time of day, etc.

The optical lens 12 may include a filter for attenuating a particular wavelength range of light (e.g., blue light).

The device 10 may additionally include a thermal management system. For example, the thermal management system may include a heat sink thermally connected to the circuitry 18 and/or light source 14. The heat sink may be located on an exterior of the device 10.

The device 10 may also include a power management system configured to optimize battery life. For example, the circuitry 18 may be operated to reduce heat generation and/or reduce electrical power usage based on a temperature of the device 10 and/or a remaining battery life. For example, functions unrelated to delivery of light therapy may be reduced or turned off based on battery life.

The device 40 may additionally include energy harvesting. For example, the device 10 may include an electricity generated for charging a power supply 70 from light or motion of the device 10. For example, the electricity generated may include at least one of piezoelectric or photovoltaics.

As described above, the device 10 may be configured to communicate with external electronic devices. For example, the device 10 may include a communication interface for communicating with internet of things (IOT) devices.

The device 10 may also include a fall detector. For example, the fall detector may comprise an accelerometer or a gyroscope for detecting when a user of the device 10 falls. The device 10 may notify a third party upon detecting a fall.

The optical lens 12 may act as optical coatings for augmented reality (AR) or wavelength filtering. For example, the lens 12 may act as a screen that receives images from a camera for displaying.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the invention has been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the invention. In addition, while a particular feature of the invention may have been described above with respect to only one or more of several illustrated embodiments, such feature may be combined with one or more other features of the other embodiments, as may be desired and advantageous for any given or particular application.

1. A photobiomodulation (PBM) device for delivering phototherapy to an eye of a user, the PBM device comprising:
an optical lens;
a light source configured to emit electromagnetic radiation;
frames configured to support the optical lens and the light source relative to the eye, wherein:
the optical lens is configured to receive the emitted electromagnetic radiation along an edge of the optical lens and to propagate the electromagnetic radiation within the optical lens via total internal reflection; and
the optical lens includes light-extracting features configured to extract the propagated electromagnetic radiation from the optical lens, such that the extracted electromagnetic radiation is directed in a pre-determined light distribution;
a photosensor configured to detect a property of ambient light; and
circuitry configured to:
determine an applied optical dose delivered by the extracted electromagnetic radiation; and
control the emission of the electromagnetic radiation by the light source based on the determined applied optical dose, a defined optical dose, and the detected properties of the ambient light.

2. (canceled)

3. The PBM device of claim 1, wherein:
the detected property of the ambient light includes an intensity of the ambient light; and
when the detected intensity of the ambient light is below an ambient light intensity threshold, the emission of the electromagnetic radiation is controlled such that an intensity of the emitted electromagnetic radiation is reduced below a therapeutic light intensity.

4. The PBM device of claim 1, wherein the applied optical dose is determined based on at least one of the time duration, intensity, and wavelength of the electromagnetic radiation.

5. The PBM device of claim 1, further comprising a photosensor configured both to receive a portion of the emitted electromagnetic radiation and to detect a property of the received portion of the electromagnetic radiation, wherein the circuitry determines the applied optical dose based on the detected property.

6. The PBM device of claim 5, wherein the detected property includes at least one of wavelength or intensity.

7. The PBM device of claim 1, further comprising a monitoring sensor configured to output a sensor measurement, wherein:
the monitoring sensor includes at least one of a current sensor or a temperature sensor;
the current sensor outputs a current measurement as the sensor measurement based on a current supplied to the light source;
the temperature outputs a temperature measurement as the sensor measurement based on at least one of a temperature of the light source or a temperature of the eye of the user;
the circuitry is configured to at least one of:
determine the applied optical dose based on the sensor measurement; or
receive the sensor measurement, determine whether the sensor measurement is within an acceptable range, and issue a notification when the sensor measurement is not within the acceptable range.

8. The PBM device of claim 1, wherein:
the optical lens has an optical axis passing through a center of curvature of the optical lens;
the frames are configured to position the optical lens relative to the eye such that the optical axis intersects with a retina of the eye;
the light-extracting features are positioned away from the optical axis, such that the electromagnetic radiation extracted by the light-extracting features illuminates a peripheral portion of the eye away from a fovea centralis of the retina.

9. The PBM device of claim 8, wherein the frames include:
a bridge having nose pads configured to interact with a nose of the user when the frames are positioned on a face of the user;
two rims including a support rim, wherein each of the two rims is attached to the bridge and the support rim supports the optical lens; and
two temples, wherein each temple extends from one of the two rims and is configured to interact with an ear of the user when the frames are positioned on the face of the user.

10. The PBM device of claim 9, wherein the light source is supported by the support rim adjacent to an edge of the optical lens, such that the electromagnetic radiation emitted by the light source is received by the edge of the optical lens.

11. The PBM device of claim 9, wherein:
the electromagnetic radiation emitted by the light source is received by a light guide and is transported by the light guide to an edge of the optical lens; and
the light guide emits the transported electromagnetic radiation, such that the electromagnetic radiation is received by the edge of the optical lens.

12. The PBM device of claim 1, wherein the light source includes multiple light emitters.

13. The PBM device of claim 12, wherein:
the optical lens includes a right lens and a left lens;
the light source includes four right lens light emitters and four left lens light emitters;
the four right lens light emitters include a left side pair of light emitters and a right side pair of light emitters; and
the four left lens light emitters include a left side pair of light emitters and a right side pair of light emitters.

14. The PBM device of claim 12, wherein the light emitters include at least one of a light emitting diode or laser diode.

15. The PBM device of claim 1, wherein the optical lens includes a right lens and a left lens.

16. The PBM device of claim 1, wherein the light-extracting features include at least one of micro-lenses, reflective spots, partial reflective planes, diffraction gratings.

17. The PBM device of claim 16, wherein:
the light-extracting features includes a micro-lens array;
the light source includes light emitters; and
the light emitters are mounted to a flexible printed circuit.

18. The PBM device of claim 1, wherein the light source emits electromagnetic radiation in a wavelength range of 500 nm to 1000 nm.

19. The PBM device of claim 1, wherein the defined optical dose includes an illuminance of 0.4-0.8 mW/cm2 at a cornea of the eye of the user.

20. A photobiomodulation (PBM) system for delivering phototherapy to an eye of a user, the PBM system comprising:
the PBM device of claim 1, further comprising a power storage device physically supported by the frames and configured both to store electrical power and to supply the stored electrical power to the circuitry and the light source; and
a charger configured to receive the frames of the PBM device and, when the frames are received by the charger, supply the electrical power to the power storage device.

21. The PBM system of claim 20, wherein:
the charger includes a controller and a photodetector;
the controller is configured to cause the light source to emit the electromagnetic radiation;
the photodetector is configured to receive the electromagnetic radiation extracted from the optical lens;
the controller is further configured to:
determine properties of the electromagnetic radiation received by the photodetector; and
determine whether the determined properties are consistent with the pre-determined light distribution.

22. The PBM system of claim 21, wherein the controller is configured to determine a measured light distribution based on the electromagnetic radiation received by the photodetector.

23. The PBM system of claim 22, wherein the measured light distribution includes an angular output of the electromagnetic radiation from the optical lens.

24. The PBM system of claim 22, wherein the photosensor is positioned relative to the optical lens, such that when the frames are positioned on the charger, the position of the photosensor matches a location of a defined structure of the eye when the frames are positioned on the face of the user.

25. The PBM system of claim 22 wherein, when the measured light distribution is inconsistent with the pre-determined light distribution, the controller is further configured to at least one of:
issue a miscalibration notification; or
perform calibration of the light source until the measured light distribution is consistent with the pre-determined light distribution by repeatedly:
determining recalibration parameters based on the measured light distribution and the pre-determined light distribution;
issuing the recalibration parameters to the circuitry;
causing the light source to emit electromagnetic radiation based on the issued recalibration parameters, such that the photodetector receives the electromagnetic radiation extracted from the optical lens;
determining the measured light distribution based on the electromagnetic radiation received by the photodetector; and
determining whether the measured light distribution is consistent with the pre-determined light distribution.

26. The PBM system of claim 21, wherein the charger includes:
a central support configured to physically support the optical lens when the frames are supported by the charger;
a left lateral support configured to support a first temple of the frames when the frames are supported by the charger; and
a right lateral support configured to support a second temple of the frames when the frames are supported by the charger, wherein the first temple is different from the second temple.

27. A charger for delivering electricity to a photobiomodulation (PBM) device having frames, a light source, optical lens, and circuitry, and that is configured to supply phototherapy to an eye of a user according to a pre-determined light distribution, the charger comprising:
a housing configured to receive the frames of the PBM device;
charging circuits configured to supply the electrical power to the PBM device when the frames are received by the charger;
a controller configured to cause the light source to emit electromagnetic radiation;
a photodetector configured to receive the emitted electromagnetic radiation that is extracted from the optical lens;
wherein the controller is further configured to:
determine properties of the electromagnetic radiation received by the photodetector; and
determine whether the determined properties are consistent with the pre-determined light distribution.

28. The charger of claim 27, wherein the controller is configured to determine a measured light distribution based on the electromagnetic radiation received by the photodetector.

29. The charger of claim 28, wherein the measured light distribution includes an angular output of the electromagnetic radiation from the optical lens.

30. The charger of claim 27, wherein the photosensor is supported by the housing at a positioned relative to the optical lens, such that when the frames are positioned on the charger, the position of the photosensor matches a location of a defined structure of the eye when the frames are positioned on the face of the user.

31. The charger of claim 25 wherein, when the measured light distribution is inconsistent with the pre-determined light distribution, the controller is further configured to at least one of:
issue a miscalibration notification; or
perform calibration of the light source until the measured light distribution is consistent with the pre-determined light distribution by repeatedly:
determining recalibration parameters based on the measured light distribution and the pre-determined light distribution;
issuing the recalibration parameters to the circuitry;
causing the light source to emit electromagnetic radiation based on the issued recalibration parameters, such that the photodetector receives the electromagnetic radiation extracted from the optical lens;
determining the measured light distribution based on the electromagnetic radiation received by the photodetector; and
determining whether the measured light distribution is consistent with the pre-determined light distribution.

32. The charger of claim 27, wherein the housing includes:
a central support configured to physically support nose pads of a bridge of the frames when the frames are supported by the charger;
a left lateral support configured to support a first temple of the frames when the frames are supported by the charger; and
a right lateral support configured to support a second temple of the frames when the frames are supported by the charger, wherein the first temple is different from the second temple.

33. A method for treating eye trauma using a photobiomodulation (PBM) device, having a light source, optical lens, frames, and circuitry, to deliver phototherapy to an eye of a user, the method comprising:
emitting electromagnetic radiation with the light source;
receiving the emitted electromagnetic radiation along an edge of the optical lens;
propagating the electromagnetic radiation within the optical lens via total internal reflection; and
extracting the propagated electromagnetic radiation from the optical lens using light-extracting features of the optical lens, such that the extracted electromagnetic radiation is directed in a pre-determined light distribution; and
determining with the circuitry an applied optical dose delivered by the extracted electromagnetic radiation; and
controlling the emission of the electromagnetic radiation by the light source based on both the determined applied optical dose and a defined optical dose.

34. The method of claim 33, wherein the applied optical dose is determined based on at least one of the time duration, intensity, and wavelength of the electromagnetic radiation.

35. The method of claim 33, further comprising:
outputting a sensor measurement with a monitoring sensor, wherein the monitoring sensor outputs at least one of:
a current measurement as the sensor measurement based on a current supplied to the light source; or
a temperature measurement as the sensor measurement based on at least one of a temperature of the light source or a temperature of the eye of the user;
using the circuitry to at least one of:
determine the applied optical dose based on the sensor measurement; or
receive the sensor measurement, determine whether the sensor measurement is within an acceptable range, and issue a notification when the sensor measurement is not within the acceptable range.

36. The method of claim 33, wherein the electromagnetic radiation extracted by the light-extracting features illuminates a peripheral portion of the eye away from a fovea centralis of the retina.

37. The method of claim 33, further comprising:
receiving the frames on a charger;
supplying electrical power to the PBM device with the charger;
a controller of the charger causing the light source to emit the electromagnetic radiation;
receiving with a photodetector the electromagnetic radiation extracted from the optical lens;
determining properties of the electromagnetic radiation received by the photodetector using the controller; and
determining using the controller whether the determined properties are consistent with the pre-determined light distribution.

38. The method of claim 37, wherein the determined property includes a measured light distribution of electromagnetic radiation.

39. The method of claim 38, further comprising:
when the measured light distribution is inconsistent with the pre-determined light distribution, at least one of:
issuing a miscalibration notification with the controller; or
performing calibration of the light source until the measured light distribution is consistent with the pre-determined light distribution by repeatedly:
determining with the controller recalibration parameters based on the measured light distribution and the pre-determined light distribution;
issuing the recalibration parameters to the circuitry;
causing the light source to emit electromagnetic radiation based on the issued recalibration parameters, such that the photodetector receives the electromagnetic radiation extracted from the optical lens;
determining with the controller the measured light distribution based on the electromagnetic radiation received by the photodetector; and
determining with the controller whether the measured light distribution is consistent with the pre-determined light distribution.

40. The method of claim 33, wherein the trauma is caused by at least one of diabetic retinopathy, macular degeneration, or diabetic macular edema.
